SEQUENCE LISTING (9 sequences)

SEQ ID NO: 1 - protein, 229 aa, Tardigrade sp.
MSAEAMNMNMNQDAVFIPPPEGEQYERKEKQEIQQTSYLQSQVKVPLVNLPAPFFSTSFSAQEILGEGFQASISRISAVSEELSSIEIPELAEEARRDFAAKTREQEMLSANYQKEVERKTEAYRKQQEVEADKIRKELEKQHLRDVEFRKDIVEMAIENQKKMIDVESRYAKKDMDRERVKVRMMLEQQKFHSDIQVNLDSSAAGTETGGQVVSESQKFTERNRQIKQ

SEQ ID NO: 2 - protein, 19 aa, synthetic construct
YRKQQEVEADKIRKELEKQ

SEQ ID NO: 3 - protein, 19 aa, synthetic construct
ARKQQEVEADKIRKELEKQ

SEQ ID NO: 4 - protein, 19 aa, synthetic construct
YRKQAEVEADKIRKELEKQ

SEQ ID NO: 5 - protein, 19 aa, synthetic construct
YRKQQAVEADKIRKELEKQ

SEQ ID NO: 6 - protein, 19 aa, synthetic construct
YRKQQEAEADKIRKELEKQ

SEQ ID NO: 7 - protein, 19 aa, synthetic construct
YRKQQEVEADKIAKELEKQ

SEQ ID NO: 8 - protein, 19 aa, synthetic construct
YRKQQEVEADKIRKEAEKQ

SEQ ID NO: 9 - protein, 20 aa, synthetic construct
XRKQXXXVEADXIRKEXEKQ

(19) United States
(12) Patent Application Publication
(10) Pub. No.: US 2026/0285918 A1
(43) Pub. Date: Sep. 24, 2026

(54) PEPTIDE FOR INHIBITING PROTEIN AGGREGATION AND PROTECTING PROTEIN ACTIVITY

(71) Applicant: GWANGJU INSTITUTE OF SCIENCE AND TECHNOLOGY, Gwangju (KR)

(72) Inventors: DONG UK KANG, Jeonju-si Jeonbuk-do (KR); CHIN JU PARK, Gwangju (KR)

(21) Appl. No.: 19/438,019

(22) Filed: Dec. 31, 2025

Related U.S. Application Data
(63) Continuation-in-part of application No. PCT/KR2024/009393, filed on Jul. 3, 2024.

(30) Foreign Application Priority Data
Jul. 4, 2023 (KR) 10-2023-0086467

(51) Int. Cl. C07K 14/435; A61K 47/42

(52) U.S. Cl. CPC C07K 14/43504; A61K 47/42

(57) ABSTRACT

The present invention utilizes CAHS (Cytosolic Abundant Heat Soluble protein) overexpressed by the water bear (scientific name: Tardigrade), a living organism that can survive in extreme environments (high temperature: 151° C., low temperature: −273° C., vacuum, and radiation environment) to increase protein stability. In other words, the present invention is an invention that improves the physical properties while maintaining the effect of stabilizing other proteins by specifying a core sequence of the CAHS protein, so that it can be used under various conditions.

### CROSS-REFERENCE TO RELATED APPLICATIONS

This application claims priority to PCT/KR2024/009393, filed on Jul. 3, 2024, which claims priority to KR10-2023-0086467 filed Jul. 4, 2023, all of which are hereby incorporated by reference herein entirely.

### REFERENCE TO AN ELECTRONIC SEQUENCE LISTING

The content of the electronic sequence listing named POPB242234March26.xml; Size: 9,321 bytes; Date of Creation: Mar. 17, 2026, is herein incorporated by reference in its entirety.

### TECHNICAL FIELD

The present invention relates to a peptide for inhibiting protein aggregation and protecting protein activity.

### BACKGROUND ART

Biologics, such as antibodies composed of proteins, and enzymes used industrially are often unstable. This reduces product stability and increases product cost due to storage expenses at ultra-low temperatures (−80° C.). To address this, excipients are added and formulations are modified to stabilize the product; however, storage at low temperatures remains necessary. Trehalose, a type of excipient found in organisms resistant to dry environments, can raise the temperature at which proteins denature. However, it has side effects such as reducing drug efficacy or inducing autophagy. Attempts have been made to protect protein-based drugs and enzymes using recombinant human serum albumin (rhSA), but even with this, storage at extremely low temperatures (−80° C.) remains necessary. The issues with conventional technology can be summarized as follows: First, the cost of ultra-low temperature storage increases the price of protein-based drugs and enzymes. Second, trehalose, a known excipient, can potentially inhibit drug efficacy. Third, even when using recombinant human serum albumin, a known excipient, ultra-low temperature storage remains necessary, failing to reduce pharmaceutical storage costs.

### Technical Problem

An object of the present invention is to provide a composition for stabilizing biological materials containing CAHS (Cytosolic Abundant Heat Soluble protein).

Another object of the present invention is to provide a method for stabilizing biological materials, comprising the step of adding a composition for stabilizing biological materials containing CAHS (Cytosolic Abundant Heat Soluble protein) protein to the biological materials.

Another object of the present invention is to provide a pharmaceutical formulation comprising the composition for stabilizing biological materials, and a pharmaceutical formulation containing the target biological material.

More specifically, the object of the present invention is to provide a composition for stabilizing biological materials that not only protects the activity of a target protein to a higher level than existing excipients even when a smaller amount is added by specifying a certain region of a CAHS (Cytosolic Abundant Heat Soluble protein), but also has improved physical properties.

However, the technical problem to be achieved by the present invention is not limited to the above-mentioned problems, and other problems that are not mentioned will be clearly understood by a skilled person in the art from the following description.

### Solution to Problem

Various embodiments of the present invention are described with reference to the accompanying drawings. In the following description, various specific details, such as specific forms, compositions, and processes, are described for a complete understanding of the present invention. However, specific embodiments may be implemented without one or more of these specific details or in combination with other known methods and forms. In other examples, known processes and manufacturing techniques are not described in detail to avoid making the present invention unnecessary or ambiguous. A reference throughout this specification to a single embodiment means that the special features, forms, compositions, or characteristics described in connection with that embodiment are included in one or more embodiments of the present invention. Therefore, the situations of embodiments described at various locations throughout this specification do not necessarily represent the same embodiments of the invention. Additionally, special features, forms, compositions, or characteristics may be combined in any suitable manner in one or more embodiments.

Unless specifically defined herein, all scientific and technical terms used in this specification shall have the same meanings as commonly understood by those skilled in the art to which the present invention belongs.

### 1. Composition for Stabilizing Biological Materials Containing CAHS (Cytosolic Abundant Heat Soluble Protein) Protein

According to an embodiment of the present invention, a composition for stabilizing biological material containing CAHS (Cytosolic Abundant Heat Soluble protein) is provided.

In the present invention, the “CAHS (Cytosolic Abundant Heat Soluble protein) protein” or “CAHS protein” refers to a type of, a specific protein found in tardigrades (scientific name: Tardigrade), representing a particular type of intrinsically disordered protein observed in tardigrades. They are most commonly used to help tardigrades survive in dry conditions, conferring extreme tolerance. Due to the flexibility of these proteins, CAHS proteins are highly influenced by the environment, undergoing significant changes in extreme abiotic conditions. Furthermore, the protective mechanism in tardigrades was originally thought to be due to high trehalose levels. Trehalose has been shown to work alongside heat shock proteins, aiding dehydration-sensitive proteins and helping organisms like yeast survive desiccation by maintaining them in solution. However, when tested in tardigrades, trehalose levels were found to be low or undetectable, making this theory controversial in tardigrades. Trehalose appears to accumulate at low or undetectable levels in tardigrades, insufficient to provide protection. Tardigrades require tardigrade-specific proteins, including CAHS proteins, to survive. These tardigrade-specific proteins are a type of intrinsically disordered protein. This means they lack a defined three-dimensional structure, unlike conventional proteins that rely on folding to perform specific tasks. These proteins utilize various forms, called ensembles, to exploit different structures. Because of this, IDPs (intrinsically disordered proteins) are highly likely to respond strongly to their environment. Furthermore, there are three families of tardigrade-specific proteins, each named after the location within the cell where the protein is found. These three families include cytoplasmic-, secreted-, and mitochondrial-abundant heat-soluble (CAHS, SAHS, and MAHS) proteins, collectively termed Tardigrade Disordered Proteins (TDP) or Tardigrade-specific proteins. At the sequence conservation level, TDPs are unique to tardigrades, and the CAHS, SAHS, and MAHS families, beyond their high levels of disorder, are not similar to one another. All three families are structurally highly expressed or become significantly abundant in response to desiccation. In vivo, in vitro, and in situ experiments demonstrate the functional role of each TDP family member in mitigating cellular disruption induced by various abiotic stresses. These proteins resemble abundant proteins in late embryonic development, except for their specificity to tardigrades. The three families are not homologous and have been shown to be expressed or upregulated during desiccation. Unlike conventional proteins, intrinsically disordered proteins do not precipitate or denature in solution during high heating. Tardigrades rely on these proteins to survive in extreme environments, enabling them to endure many abiotic factors such as freezing and heat. Dehydration causes problems for cells, where proteins typically depend on a hydrated environment to perform many functions, but tardigrade-specific proteins help prevent contents from clumping when first dehydrated and maintain membrane integrity during rehydration.

Recent research findings indicate that when comparing the sequences of eight CAHS (Cytosolic Abundant Heat Soluble protein) proteins (CAHS1, CAHS2, CAHS3 from R. varieornatus, and five proteins from H. dujardini and M. tardigradum), conserved domains were identified as HTD/E, ASAARIS, CAHS-c1, and CAHS-c2. Within the CAHS-c2 region, two 19-mer motifs, CAHS-motif 1 and CAHS-motif 2, were reported to be conserved. However, it remains unknown which of these diverse conserved regions influences the core function of CAHS proteins.

Furthermore, water is often considered essential for life since it is required for all metabolic processes. However, many organisms across all biological kingdoms challenge this notion by surviving despite losing all or nearly all of the bulk water within their bodies and cells. To achieve this, specialized organisms enter a state of suspended animation known as anhydrobiosis 1,2,3. In this anhydrobiotic state, the desiccated organism can persist for weeks, years, or in extreme cases, centuries, and upon rehydration, can reactivate its metabolism and resume life processes. One such anhydrobiotic organism is the tardigrade, colloquially known as the water bear. Tardigrades are microscopic, eight-legged animals phylogenetically related to arthropods and nematodes. Currently, ~1400 species of tardigrades have been described, many of which can withstand numerous abiotic stresses, including desiccation. How tardigrades survive desiccation remains one of the enduring mysteries of organismal physiology. Biochemical and molecular studies have begun to unravel how tardigrades survive desiccation, implicating antioxidant enzymes and reactive oxygen species scavengers, storage proteins, and tardigrade-specific disordered proteins, as well as DNA repair mechanisms that mediate desiccation resistance. This pioneering research has been significantly enhanced by the adoption of various-omics approaches, primarily stemming from next-generation sequencing of genomes and transcriptomes from diverse tardigrade species. While gene and transcriptome complementation in tardigrades under desiccation conditions has been evaluated to some extent, analyses at other-omics levels, such as metabolomics, remain scarce for tardigrades.

In the present invention, the “biological material” may be selected from the group consisting of DNA, RNA, peptides, proteins, polysaccharides, and polypeptides, or mixtures thereof. More specifically, it may be a protein or a nucleic acid. Preferred peptides and proteins include enzymes, antibodies, blood coagulation factors, insulin, interferon, hormones, cytokines, transcription factors, and other regulatory proteins or fragments thereof, or mixtures thereof. Nucleic acids (DNA and RNA) may be single-stranded or double-stranded, high molecular weight, low molecular weight or short molecules (e.g., miRNA, siRNA, or highly degraded nucleic acids), specifically represented by viral RNA, bacterial RNA, fungal RNA, or cellular RNA, PCR products, genomic DNA or cDNA, viral DNA or cDNA, bacterial DNA or cDNA, fungal DNA or cDNA, plasmid DNA or cDNA. New biological material includes supramolecular structures (e.g., viral particles (e.g., adenovirus, adeno-associated virus, retrovirus, lentivirus, varicella-zoster virus, HIV, or herpesvirus), cells of prokaryotic organisms, or cells of eukaryotic organisms). The aforementioned biological material may be collected from natural sources or biotechnically engineered sources, or synthesized by techniques such as recombinant technology and chemical synthesis.

In another embodiment of the present invention, the CAHS protein provides a composition for stabilizing biological material, as represented by SEQ ID NO: 1.

In another embodiment of the present invention, the CAHS protein is the one represented by SEQ ID NO: 2, providing a composition for stabilizing biological material.

The CAHS protein represented by SEQ ID NO: 2 in the present invention is the protein corresponding to the sequence from the 124th to the 142nd position within the entire CAHS protein sequence, also referred to as CAHS motif 1 ₁₂₄₋₁₄₂ in the specification at. Furthermore, the CAHS protein plays various roles enabling tardigrades to survive in dry conditions, high temperatures, low temperatures, vacuum, and radiation environments. In particular, the CAHS protein represented by SEQ ID NO: 2 corresponds to the protein for the sequence from the 124th to the 142nd position within the entire CAHS protein sequence. This portion is likely an essential part enabling tardigrades to survive in such extreme environments (dry conditions, high temperatures, low temperatures, vacuum, and radiation environments).

In another embodiment of the present invention, a composition for stabilizing a biological material is provided, wherein the protein comprises at least one selected from the group consisting of growth factors, hormones, interferons, vaccines, recombinant proteins, recombinant peptides, and enzymes.

In another embodiment of the present invention, the biological material stabilization provides a composition for stabilizing a biological material, wherein the biological material stabilization prevents denaturation or aggregation of the biological material.

In the present invention, the terms “denaturation” or “denatured” refer to the loss of the secondary, tertiary, or quaternary structure present in its native state due to exposure to certain external stimuli or compounds, such as strong acids or bases, heat, concentrated inorganic salts, organic solvents (alcohol or chloroform), or radiation) causes the loss of its native secondary, tertiary, or quaternary structure. Denaturation of proteins in living cells leads to disruption of cellular activity and cell death. Protein denaturation is a consequence of cell death. Denatured proteins can exhibit a wide range of characteristics, from conformational changes and loss of solubility to aggregation due to exposure of hydrophobic groups. Denatured proteins cannot function because they lose their three-dimensional structure.

In the present invention, the term “aggregation” refers to the phenomenon where biological materials, such as essentially disordered or misfolded proteins, aggregate (i.e., accumulate and clump together) within or outside cells. Protein aggregates are implicated in various diseases known as amyloidosis, including ALS, Alzheimer's disease, Parkinson's disease, and prion diseases. Furthermore, after synthesis, proteins typically fold into a specific three-dimensional structure that is thermodynamically most favorable. Such protein structures are stabilized by non-covalent interactions between two cysteine residues and disulfide bonds. Non-covalent interactions include ionic interactions and weak van der Waals interactions. Ionic interactions form between anions and cations, creating salt bridges that help stabilize the protein. Van der Waals interactions include nonpolar interactions (i.e., London dispersion forces) and polar interactions (i.e., hydrogen bonding, dipole-dipole interactions). These play crucial roles in the secondary and tertiary structures of proteins, such as the formation of alpha helices or beta sheets. Interactions between amino acid residues within a specific protein are crucial for its final structure. When noncovalent interactions change, as can occur with alterations in the amino acid sequence, the protein becomes prone to misfolding or unfolding. In such cases, if the cell fails to assist in refolding the protein or degrade the unfolded protein, the unfolded/misfolded protein may aggregate. Here, the exposed hydrophobic regions of the protein can interact with the exposed hydrophobic patches of other proteins.

In another embodiment of the present invention, a composition for stabilizing biological material is provided, wherein the denaturation or aggregation of the biological material occurs during a dehydration-rehydration process or a freeze-thaw process.

### 2. Method for Stabilizing Biological Material Containing CAHS (Cytosolic Abundant Heat Soluble Protein)

In one embodiment of the present invention, a method for stabilizing biological material is provided, comprising the step of adding a composition for stabilizing biological material containing CAHS (Cytosolic Abundant Heat Soluble protein) protein to the biological material.

The description of the CAHS protein and biological material is omitted here to avoid redundancy, as it is described in the composition for stabilizing biological material containing the CAHS (Cytosolic Abundant Heat Soluble protein) protein described in 1. above.

In another embodiment of the present invention, a method for stabilizing a biological material is provided, wherein the CAHS protein is represented by SEQ ID NO: 1. In another embodiment of the present invention, a method for stabilizing a biological material is provided, wherein the CAHS protein is represented by SEQ ID NO: 2.

In another embodiment of the present invention, a method for stabilizing a biological material is provided wherein the mass ratio of the biological material to the CAHS protein is 1:2 or greater. In another embodiment of the present invention, a method for stabilizing a biological material is provided wherein the mass ratio of the biological material to the CAHS protein is from 1:2 to 1:100.

The present invention can protect the activity of the biological material targeted in the dehydration-rehydration process at a high level when the mass ratio of the biological material to the CAHS protein is 1:2 or greater, and can protect the activity of the target biological material at a high-level during freeze-thaw cycles when the ratio of the target biological material to the CAHS protein is 1:4 or higher. Thus, the mass ratio of the target biological material to the CAHS protein is 1:2 or greater, or preferably 1:2 to 1:100, or 1:2 to 1:80, or 1:70, 1:2.5 or greater, 1:2.5 to 1:100, or 1:2.5 to 1:50, but is not limited there.

### 3. a Pharmaceutical Formulation Comprising the Composition for Stabilizing Biological Material and the Target Biological Material.

In one embodiment of the present invention, a pharmaceutical formulation is provided comprising the biological material stabilizing composition described in 1, and 2. above and the target biological material.

The description of the CAHS protein and biological material is omitted here to avoid redundancy, as it is described in the composition for stabilizing biological material containing the CAHS (Cytosolic Abundant Heat Soluble protein) protein described in 1. above.

In one embodiment of the present invention, the pharmaceutical formulation further comprises a stabilizing buffer solution.

In one embodiment of the present invention, “stabilizer” means any additive added to increase the stability of an active ingredient and prevent the active ingredient from being arbitrarily changed, oxidized, crystallized, or degraded into a compliant substance, and is not particularly limited as long as it is pharmaceutically acceptable. The evaluation of the stabilizing effect of a stabilizer can be performed without limitation on the temperature, but it is generally understood that the stabilizing effect is maintained for a long time at low temperatures rather than at high temperatures. Therefore, the evaluation of long-term stabilizing effect at low temperatures can be replaced with an “accelerated experiment” performed for a short time at high temperatures. For example, the result of evaluating the stabilizing effect of a specific stabilizer at 37° C. for 9 days can be understood to be the same as the result of evaluating at 4° C. for 3 months, and the result of evaluating at 37° C. for 74 days can be understood to be the same as the result of evaluating at 4° C. for 24 months (iso-inc.com/medical-package-testing/accelerated-aging.html).

In one embodiment of the present invention, a “stabilizing buffer solution” or “stabilizing buffer agent” refers to a substance that simultaneously possesses the effects of a stabilizer and a buffer solution. It can be manufactured by adding a substance with stabilizing effects to a general buffer solution, and including additional stabilizers in the stabilizing buffer solution can be expected to produce a synergistic stabilizing effect. In the present invention, a glucono-delta-lactone buffer solution and/or a tartaric acid buffer solution are preferred for the stabilizing buffer solution, but are not limited thereto.

In the present invention, the “prevention” may include, without limitation, any act of blocking symptoms caused by a disease, or suppressing or delaying the symptoms, using the composition of the present invention.

In the present invention, the “treatment” or “amelioration” may include, without limitation, any act capable of ameliorating or beneficially altering symptoms caused by a disease, using the composition of the present invention.

In the present invention, the pharmaceutical composition may be characterized by being in the form of capsules, tablets, granules, injections, ointments, powders, or beverages, and the pharmaceutical composition may be characterized by being targeted to humans.

The pharmaceutical composition of the present invention may be formulated in the form of oral preparations such as powders, granules, capsules, tablets, and aqueous suspensions, preparations for external use, suppositories, and sterile injectable solutions, respectively, according to conventional methods, and used. However, the pharmaceutical composition is not limited thereto. The pharmaceutical composition of the present invention may further comprise a pharmaceutically acceptable carrier. As the pharmaceutically acceptable carrier, a binder, a glidant, a disintegrant, an excipient, a solubilizer, a dispersant, a stabilizer, a suspending agent, a pigment, a flavor, and the like may be used for oral administration; a buffer, a preserving agent, a pain-relieving agent, a solubilizer, an isotonic agent, a stabilizer, and the like may be used in admixture for injections; and a base, an excipient, a lubricant, a preserving agent, and the like may be used for topical administration. The preparations of the pharmaceutical composition of the present invention may be prepared in various ways by being mixed with the pharmaceutically acceptable carrier as described above. For example, for oral administration, the pharmaceutical composition may be formulated in the form of tablets, troches, capsules, elixirs, suspensions, syrups, wafers, or the like. For injections, the pharmaceutical composition may be formulated in the form of unit dosage ampoules or multiple dosage forms. Alternatively, the pharmaceutical composition may be formulated into solutions, suspensions, tablets, capsules, sustained-release preparations, or the like.

Meanwhile, as examples of carriers, diluents, or excipients suitable for making preparations, lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, or the like may be used. In addition, a filler, an anti-coagulant, a lubricant, a wetting agent, a fragrance, an emulsifier, a preservative, and the like may further be included.

The route of administration of the pharmaceutical composition according to the present invention includes, but is not limited to, oral, intravenous, intramuscular, intraarterial, intramedullary, intradural, intracardiac, transdermal, subcutaneous, intraperitoneal, intranasal, intestinal, topical, sublingual, or rectal route. Oral or parenteral administration is preferred.

In the present invention, the “parenteral” includes subcutaneous, intradermal, intravenous, intramuscular, intraarticular, intrabursal, intrasternal, intradural, intralesional, and intracranial injection or infusion techniques. The pharmaceutical composition of the present invention may also be administered in the form of suppositories for rectal administration.

The pharmaceutical composition of the present invention may vary depending on a variety of factors, including activity of a certain compound used, the patient's age, body weight, general health status, sex, diet, frequency of administration, route of administration, rate of excretion, drug combination, and severity of a certain disease to be prevented or treated. A dose of the pharmaceutical composition may vary depending on the patient's condition, body weight, severity of disease, drug form, route of administration, and duration, and may be appropriately selected by those skilled in the art. The pharmaceutical composition may be administered in an amount of 0.0001 to 50 mg/kg or 0.001 to 50 mg/kg, per day. Administration may be made once a day or several times a day. The dose is not intended to limit the scope of the invention in any way. The pharmaceutical composition according to the present invention may be formulated in the form of pills, sugar-coated tablets, capsules, liquids, gels, syrups, slurries, or suspensions.

The food composition that includes the composition of the present invention may be prepared in the form of various foods, for example, beverages, gums, tea, vitamin complexes, powders, granules, tablets, capsules, confections, rice cakes, bread, and the like. The food composition of the present invention is composed of a plant extract having little toxicity and side effects, and thus can be used without worries in a case of being ingested for a long time for preventive purposes.

When the composition of the present invention is included in the food composition, it may be added in an amount corresponding to a rate of 0.1% to 50% of the total weight.

Here, in a case where the food composition is prepared in the form of beverages, there is no particular limitation except that the beverage contains the food composition at an indicated proportion, and the beverage may contain various flavoring agents, natural carbohydrates, or the like as additional ingredients, similarly to conventional beverages. That is, examples of the natural carbohydrates may include monosaccharides such as glucose, disaccharides such as fructose, polysaccharides such as sucrose, conventional sugars such as dextrin and cyclodextrin, and sugar alcohol such as xylitol, sorbitol, and erythritol. Examples of the flavoring agents may include natural flavoring agents (thaumatin, stevia extracts (such as rebaudioside A), glycyrrhizin, and the like) and synthetic flavoring agents (saccharin, aspartame, and the like).

In addition, the food composition of the present invention may contain various nutrients, vitamins, minerals (electrolytes), flavorings such as synthetic flavorings and natural flavorings, colorants, pectic acid and salts thereof, alginic acid and salts thereof, organic acids, protective colloidal thickeners, pH adjusting agents, stabilizers, preservatives, glycerin, alcohol, carbonizing agents used in carbonated beverages, and the like.

These ingredients may be used individually or in combination. A proportion of such additives is not so important, and is generally selected from the range of 0.1 to about 50 parts by weight per 100 parts by weight of the composition of the present invention.

The appropriate total daily dosage may be determined by the prescribing physician within the scope of proper medical judgment and may be administered in one or more divided doses. However, for the purposes of the present invention, the specific therapeutic effective dose for a particular patient depends on factors including the type and degree of response sought, whether other formulations are used in certain cases, the specific composition containing the aforementioned active ingredient, the patient's age, weight, general health status, gender, and diet, the time of administration, the route of administration, the secretion rate of the composition containing the active ingredient, the duration of treatment, and various factors, including drugs used with or concurrently administered with the specific composition, as well as similar factors well known in the pharmaceutical field.

In the present invention, the term “combination” should be understood to mean simultaneous, separate, or sequential administration. If the administration is sequential or separate, the interval between the administration of the secondary component should be such that the beneficial effect of the combination is not lost.

In the present invention, the dosage of the conjugated molecule; or the antibody-drug conjugate may be approximately 0.0001 μg to 500 mg per kg of patient body weight, but is not limited thereto.

In another embodiment of the present invention, a method for stabilizing a biological material is provided, wherein the CAHS protein is represented by SEQ ID NO: 1.

In another embodiment of the present invention, a method for stabilizing a biological material is provided, wherein the CAHS protein is represented by SEQ ID NO: 2.

In another embodiment of the present invention, a method for stabilizing a biological material is provided, wherein the CAHS protein is a sequence in which one of the CAHS motif 1 sequences represented by SEQ ID NO: 2 is mutated to alanine.

In another embodiment of the present invention, a method for stabilizing a biological material is provided, wherein the CAHS protein is any one selected from the group consisting of SEQ ID NOs: 3 to 8.

In another embodiment of the present invention, a method for stabilizing a biological material is provided, wherein the mass ratio of the biological material to the CAHS protein is 1:2 or greater.

In another embodiment of the present invention, a method for stabilizing a biological material is provided, wherein the mass ratio of the biological material to the CAHS protein is 1:2 to 1:100. In another embodiment of the present invention, a method for stabilizing a biological material is provided, wherein the biological material is any one selected from the group consisting of DNA, RNA, peptides, proteins, polysaccharides, and polypeptides.

In another embodiment of the present invention, a method for stabilizing a biological material is provided, wherein the protein comprises at least one selected from the group consisting of growth factors, hormones, interferons, vaccines, recombinant proteins, recombinant peptides, and enzymes.

In another embodiment of the present invention, a method for stabilizing a biological material is provided, wherein the stabilization of the biological material prevents denaturation or aggregation of the biological material.

In another embodiment of the present invention, a method for stabilizing a biological material is provided, wherein the denaturation or aggregation of the biological material occurs during a dehydration-rehydration process or a freeze-thaw process.

In another embodiment of the present invention, a pharmaceutical formulation comprising the biological material stabilization composition and the target biological material is provided. In another embodiment of the present invention, a pharmaceutical formulation is provided wherein the mass ratio of the target biological agent to the CAHS protein is 1:2 to 1:100.

In another embodiment of the present invention, a pharmaceutical formulation is provided wherein the target biological agent is any one selected from the group consisting of DNA, RNA, peptides, proteins, polysaccharides, and polypeptides.

In another embodiment of the present invention, a pharmaceutical formulation is provided wherein the protein comprises at least one selected from the group consisting of growth factors, hormones, interferons, vaccines, recombinant proteins, recombinant peptides, and enzymes.

In another embodiment of the present invention, the pharmaceutical formulation further comprises a stabilizing buffer.

In another embodiment of the present invention, the pharmaceutical formulation is provided in a liquid form.

The present invention will now be described in detail based on the embodiments.

### Advantageous Effects of Invention

The present invention has the significant effect of enabling storage of other proteins without requiring ultra-low temperatures (−80° C.), as it improves the stability of other proteins while also enhancing the physical properties, which were a drawback of existing inventions, by selecting a specific motif from the CAHS (Cytosolic Abundant Heat Soluble protein) protein. Specifically, while existing CAHS proteins were confirmed to enhance the stability of other proteins, their poor physical properties limited their usability.

This invention identifies a key sequence within the CAHS protein. By doing so, it not only protects the activity of the target protein at lower concentrations than conventional adjuvants but also improves physical properties, enabling its use under diverse conditions.

### BRIEF DESCRIPTION OF DRAWINGS

FIG. 1 shows the full sequence of the cytosolic abundant heat-soluble protein (CAHS) and the protein sequence of motif 1 within the CAHS protein.

FIG. 2 compares the solubility properties of the full sequence of the CAHS protein and motif 1 within the CAHS protein.

FIGS. 3(a) to 3(c) compare the LDH activity protection rate after dehydration-rehydration of motif 1 within the CAHS protein, CAHS full protein ₁₋₂₂₉, and another excipient, BSA.

FIGS. 3(d) to 3(f) compare the LDH activity protection rate after freeze-thaw treatment of motif 1 within the CAHS protein, CAHS full protein ₁₋₂₂₉, and another excipient, BSA.

FIG. 4 shows the results of measuring the extent to which motif 1 of the CAHS protein, the entire CAHS protein ₁₋₂₂₉, and another excipient, BSA, inhibit LDH aggregation during (A) dehydration-rehydration and (B) freeze-thaw processes.

FIG. 5(a) is a graph showing the results of an LDH assay, demonstrating that when CAHS motif 1 according to the present invention is included at −20° C., the activity of the target protein, LDH, is well preserved for 1 to 4 weeks.

FIG. 5(b) is a graph showing the results of an LDH assay, demonstrating that when CAHS motif 1 according to the present invention is included at 20° C., the activity of the target protein, LDH, is well preserved for 1 to 4 weeks.

FIG. 5(c) is a graph showing the results of an LDH assay, demonstrating that when CAHS motif 1 according to the present invention is included at 37° C., the activity of the target protein LDH is well preserved for 1 to 4 weeks. Therefore, FIGS. 5(a) and 5(b) are graphs demonstrating that CAHS motif 1 according to the present invention not only well preserves the activity of the target protein at all temperatures, but also well preserves the activity of the target protein over time.

FIGS. 6(a) and 6(b) are LDH assay results, demonstrating whether a protein in which a portion of the CAHS motif 1 sequence provided by the present invention is converted to a different amino acid sequence preserves LDH activity during dehydration-rehydration and (B) freeze-thaw processes. Specifically, the amino acids Tyr, Gln, Glu, Val, Arg, and Leu in CAHS motif 1 were each substituted with Ala, and it was confirmed that all substitutions exhibited a protective effect on LDH activity.

### DETAILED DESCRIPTION OF INVENTION

Hereinafter, the present invention will be described in more detail by way of examples. These examples are only for describing the present invention in more detail, and it will be apparent to those skilled in the art that according to the gist of the present invention, the scope of the present invention is not limited by these examples.

### EXAMPLES

1. Selection of a Specific Motif from CAHS (Cytosolic Abundant Heat Soluble Protein) Proteins

The present invention utilizes CAHS (Cytosolic Abundant Heat Soluble protein), which is overexpressed in water bears (scientific name: Tardigrade), organisms that survive in extreme environments (high temperature: 151° C., low temperature: −273° C., vacuum, radioactive environments), to enhance protein stability. To confirm whether CAHS enhances protein stability in conditions that destabilize proteins, experiments were conducted using LDH (Lactate dehydrogenase) as a model.

The full CAHS protein ₁₋₂₂₉ containing the entire amino acid sequence (FIG. 1) has been confirmed in previous studies to protect various types of proteins. However, its use is limited due to the disadvantage of the CAHS protein itself having poor solubility. Therefore, while the CAHS protein possesses diverse functions, we identified CAHS motif 1 ₁₂₄₋₁₄₂ as a sequence predicted to be a core, conserved region where CAHS functions, and essential for survival.

### 2. Solubility Comparison

First, CAHS motif 1 ₁₂₄₋₁₄₂ was obtained via peptide synthesis. CAHS motif 1 ₁₂₄₋₁₄₂ and the entire CAHS protein ₁₋₂₂₉ were each dialysed at 4° C. for 18 hours in a buffer containing 20 mM potassium phosphate, 150 mM NaCl, pH 7.0, at a concentration of 100 μM, to compare their solubility.

Compared with the entire CAHS protein ₁₋₂₂₉, CAHS motif 1 ₁₂₄₋₁₄₂ showed improved solubility, indicating it can be used under various conditions (buffer pH, and protein concentration).

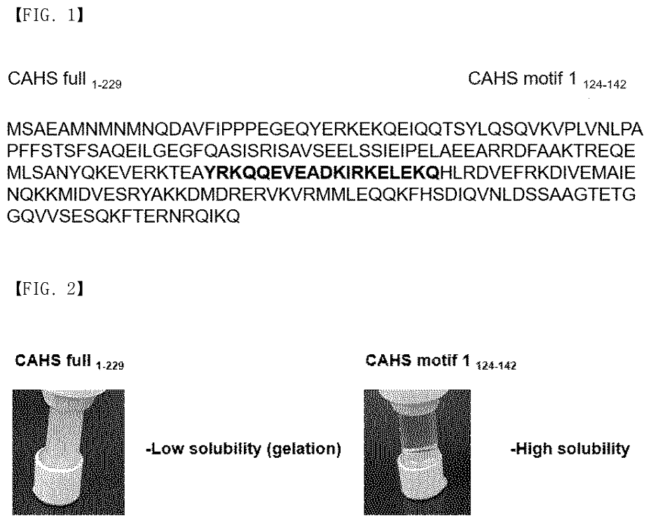

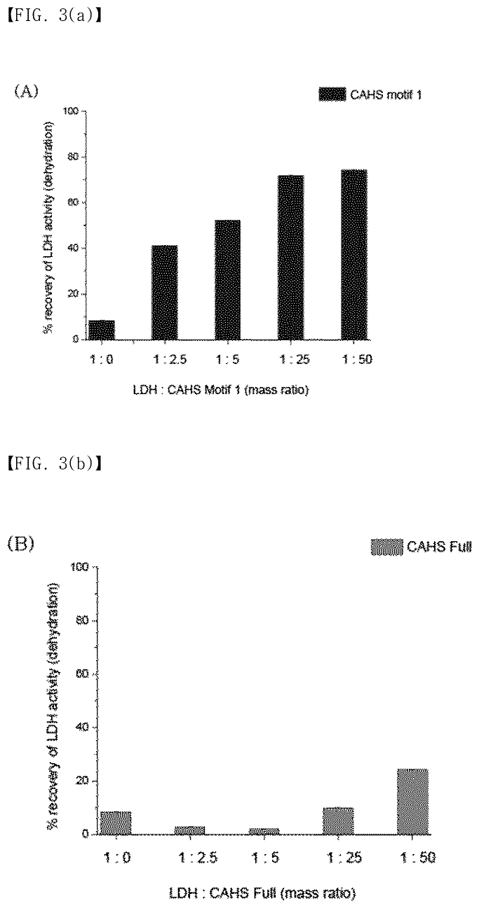

Specifically, as shown in FIG. 2, while the full-length CAHS protein ₁₋₂₂₉ exhibited poor solubility due to gelation, CAHS motif 1 ₁₂₄₋₁₄₂ according to the present invention demonstrated excellent solubility in water, showing superior solubility properties compared to the full-length CAHS protein ₁₋₂₂₉.

### 3. Comparison of Protein Activity Retention Rates

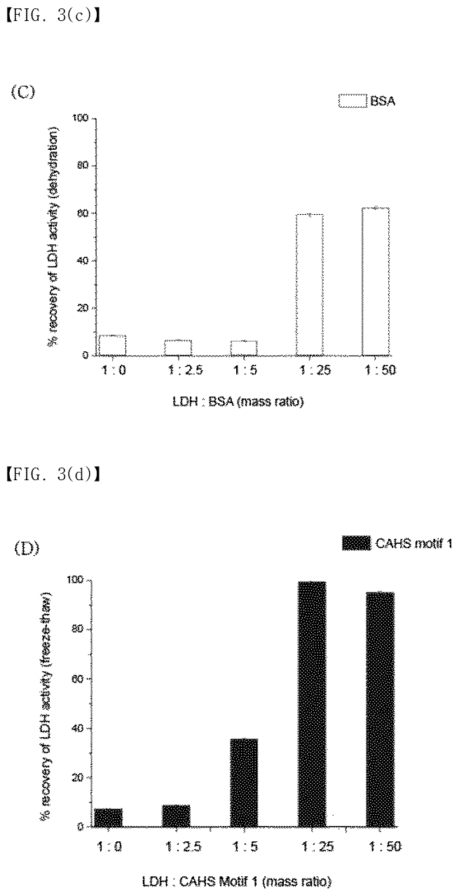

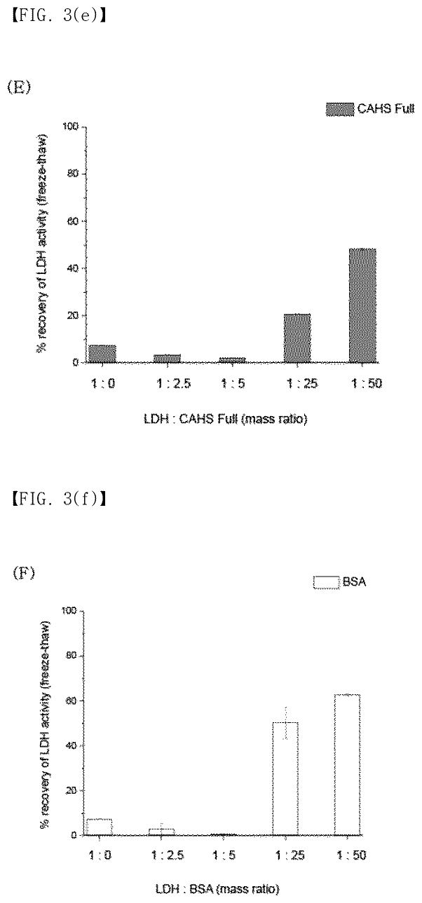

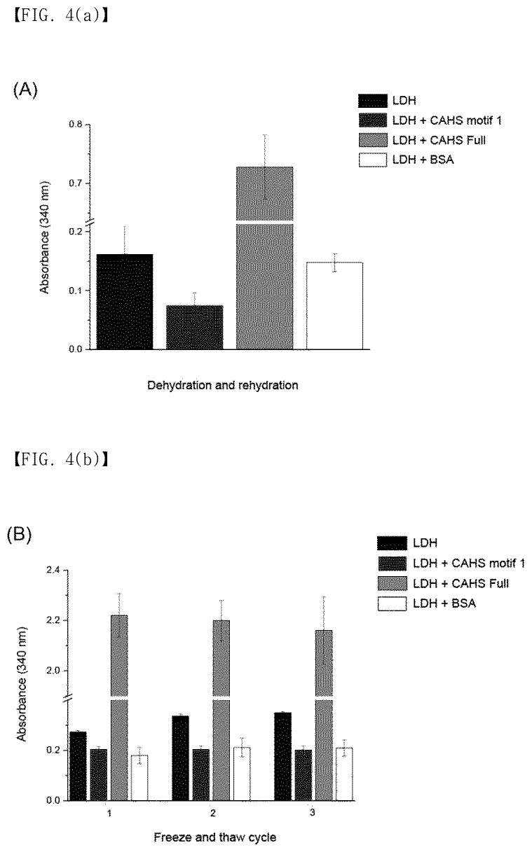

After adding the CAHS motif 1 ₁₂₄₋₁₄₂ of the present invention and the well-known excipient BSA (Bovine Serum Albumin) to the model protein LDH (Lactate dehydrogenase), dehydration-rehydration and freeze-thaw were performed to determine how much the LDH activity was protected. As a result of the experiment, it was confirmed that CAHS motif 1 ₁₂₄₋₁₄₂ protected LDH activity to a higher level than under conditions without other excipients. In addition, compared to BSA, a well-known excipient, CAHS motif 1 ₁₂₄₋₁₄₂ protected LDH activity to a higher level. Furthermore, it was confirmed that CAHS motif 1 ₁₂₄₋₁₄₂ protected LDH activity to a higher level than that of the entire CAHS protein ₁₋₂₂₉. In particular, the entire CAHS protein ₁₋₂₂₉ was not as effective as the well-known excipient BSA, confirming once again that the CAHS motif 1 ₁₂₄₋₁₄₂ best protected the activity of the protein (FIG. 3(a) to FIG. 3(f)).

Specifically, as shown in FIG. 3, the ratio of the model protein to CAHS motif 1 ₁₂₄₋₁₄₂ according to the present invention was effective even at a 1:2.5 mass ratio for dehydration-rehydration and at a 1:5 mass ratio for freeze-thaw cycles.

This is because BSA, a well-known excipient, can protect activity only when added at a mass ratio of 1:25 for dehydration-rehydration and 1:25 for freeze-thaw, and even when added at a mass ratio of up to 1/10 times less CAHS motif 1 ₁₂₄₋₁₄₂, showing a protection rate of 70-100%. Compared to BSA, a well-known excipient, which showed a protection rate of approximately 50-60%, it was confirmed that a higher activity protection rate was shown even with a smaller amount.

### 4. Comparison of Inhibition of Protein Aggregation Phenomena

After adding the CAHS motif 1 ₁₂₄₋₁₄₂ of the present invention and the well-known excipient BSA (Bovine Serum Albumin) to the model protein LDH (Lactate dehydrogenase), dehydration-rehydration and freeze-thaw were performed, and the degree of protein aggregation was confirmed through the degree of absorption at 340 nm through protein aggregation based on the light scattering effect using UV. In other words, protein aggregation is the main cause of loss of protein activity. Protein aggregation is induced during the process of freezing and thawing (freeze and thaw) or in a water-free environment (dehydration), which causes loss of protein activity. Through the present invention, it was confirmed that the CAHS motif 1 ₁₂₄₋₁₄₂ according to the present invention effectively inhibits the aggregation of LDH during the freezing and thawing process and the dehydration and rehydration process.

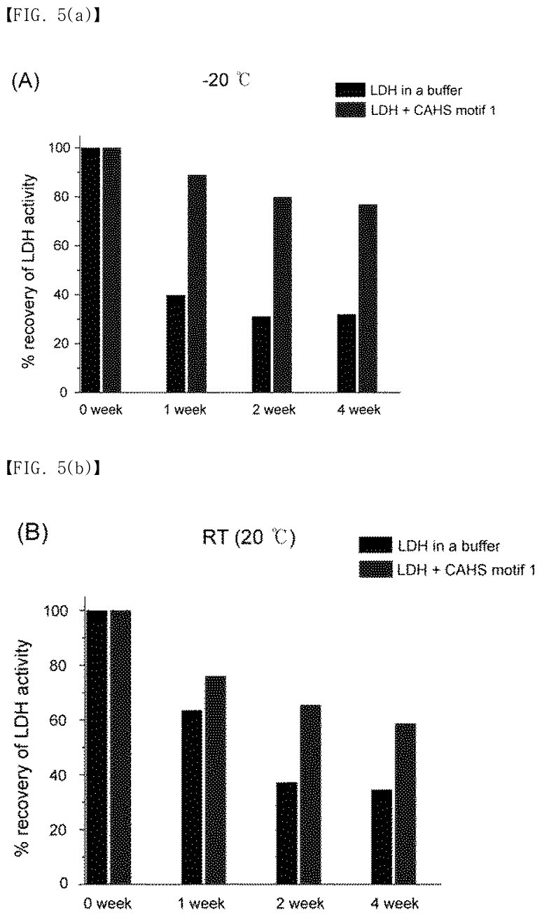

Specifically, as shown in FIG. 4, during the dehydration and rehydration process, BSA barely prevented aggregation, whereas the present invention reduced aggregation by more than half. Furthermore, FIGS. 4(a) and 4(b) show that CAHS total protein ₁₋₂₂₉ actually exhibited more severe protein aggregation under stress conditions (dehydration-rehydration and freeze-thaw).

5. Comparison of Protein Activity Protection Rates with Temperature and Time

CAHS motif 1 ₁₂₄₋₁₄₂, a stabilizer provided by the present invention, was mixed with LDH (lactate dehydrogenase), a target protein candidate for activity protection experiments, at a mass ratio of 1:50. The LDH protein activity was tested under various temperature and time conditions to determine the degree of protein activity protection.

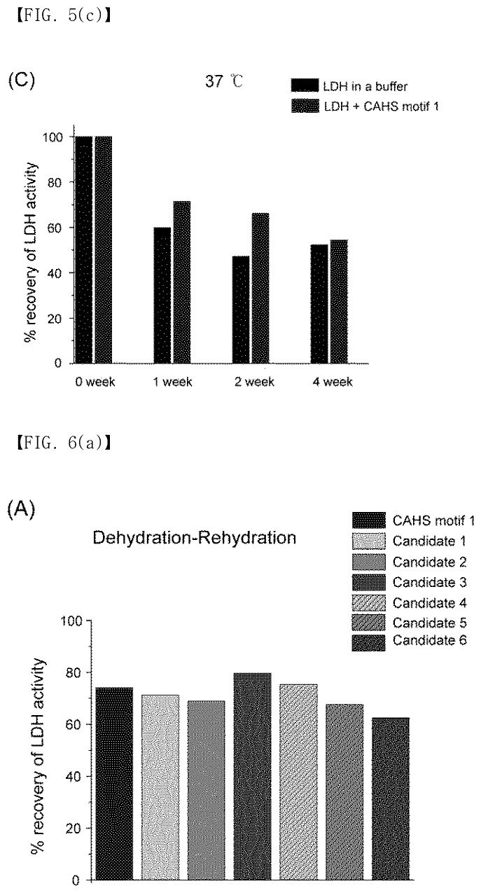

Specifically, LDH protein activity was tested at temperatures of −20° C., 20° C., and 37° C., and at time intervals of 0, 1, 2, and 4 weeks. These results are shown in FIGS. 5(a) to 5(c).

As a result, CAHS motif 1 ₁₂₄₋₁₄₂, provided by the present invention, was found to effectively preserve protein activity under all temperature and time conditions.

### 6. Comparison of Protein Activity Protection Rates for CAHS Motif 1 ₁₂₄₋₁₄₂ Sequence Variants

As shown in Table 1 below, a portion of the CAHS motif 1 ₁₂₄₋₁₄₂ sequence, a stabilizer provided by the present invention, was mutated to alanine (Ala), followed by dehydration-rehydration and freeze-thaw cycles to compare LDH activity retention efficiencies.

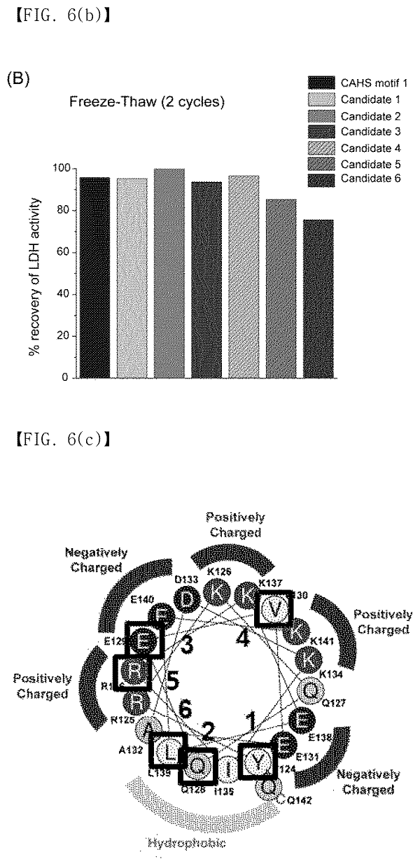

Specifically, as illustrated in FIG. 6(c), considering the charge, hydrophobicity, and hydrophilicity of each amino acid in the CAHS motif 1 ₁₂₄₋₁₄₂ sequence, Y (Tyr), Q (Gln), E (Glu), V (Val), R (Arg), and L (Leu) in the amino acid sequence of CAHS motif 1, represented by SEQ ID NO: 2, were each mutated to alanine at one site.

TABLE 1

| | Sequence | Sequence |
|---|---|---|
| Category | Information | Number |
| CAHS Motif 1 | YRKQQEVEAD | SEQ ID NO: 2 |
| | KIRKELEKQ | |
| Candidate 1 | ARKQQEVEAD | SEQ ID NO: 3 |
| (Y124A) | KIRKELEKQ | |
| Candidate 2 | YRKQAEVEAD | SEQ ID NO: 4 |
| (Q128A) | KIRKELEKQ | |
| Candidate 3 | YRKQQAVEAD | SEQ ID NO: 5 |
| (E129A) | KIRKELEKQ | |
| Candidate 4 | YRKQQEAEAD | SEQ ID NO: 6 |
| (V130A) | KIRKELEKQ | |
| Candidate 5 | YRKQQEVEAD | SEQ ID NO: 7 |
| (R136A) | KIAKELEKQ | |
| Candidate 6 | YRKQQEVEAD | SEQ ID NO: 8 |
| (L139A) | KIRKEAEKQ | |
| Conserved | XRKQXXXVEA | SEQ IN NO: 9 |
| sequence | DXIRKEXEKQ | |

As a result, as shown in FIGS. 6(a) and 6(b), it was confirmed that candidates 1 through 6 all exhibited a similar level of protective efficacy to CAHS motif 1 ₁₂₄₋₁₄₂ according to the present invention. Specifically, although the differences were not large, it was confirmed that candidates 1, 3, and 4 showed similar protective efficacy to CAHS motif 1 ₁₂₄₋₁₄₂ in dehydration-rehydration, and candidates 1, 2, 3, and 4 showed similar protective efficacy in freeze-thaw.

That is, even if the sequence is changed through the above mutations, the sequence that does not have a difference in the LDH protection effect of CAHS motif 1 is indicated by X in SEQ ID No: 9, and as a result, it was possible to confirm the sequence that plays an important role in the stabilizing function and effect of CAHS motif 1.

### CONCLUSION

1. It was confirmed that CAHS motif 1 ₁₂₄₋₁₄₂ according to the present invention protects the activity of the model protein used in the experiments better than the previously known excipient BSA during dehydration-rehydration and freeze-thaw cycles.
2. It was confirmed that CAHS motif 1 ₁₂₄₋₁₄₂ according to the present invention effectively inhibits the aggregation of the model protein LDH during dehydration-rehydration and freeze-thaw processes.
3. By specifically identifying CAHS motif 1 ₁₂₄₋₁₄₂ according to the present invention, which plays a key role in protecting protein-based materials within the entire CAHS protein 1-229, the function and solubility of the CAHS protein were improved.
4. Furthermore, it was confirmed that CAHS motif 1 ₁₂₄₋₁₄₂ provided by the present invention effectively protects protein activity under all time and temperature conditions.
5. Furthermore, it was confirmed that even when a single sequence was mutated to alanine, it effectively protected protein activity in a manner similar to the provided CAHS motif 1 ₁₂₄₋₁₄₂.

1. A composition for stabilizing a biological material comprising a CAHS (Cytosolic Abundant Heat Soluble protein) which is represented by SEQ ID NO: 9.

2. The composition according to claim 1,
wherein the CAHS protein is represented by SEQ ID NO: 2.

3. The composition according to claim 32,
wherein the CAHS protein is a protein in which one sequence of CAHS motif 1 represented by SEQ ID NO: 2 is mutated to alanine.

4. The composition according to claim 3,
wherein the CAHS protein is any one selected from the group consisting of SEQ ID NOs: 3 to 8.

5. The composition according to claim 2,
wherein the CAHS protein is represented by SEQ ID NO: 1.

6. The composition according to claim 3,
wherein the biological material is any one selected from the group consisting of DNA, RNA, peptides, proteins, polysaccharides, and polypeptides.

7. The composition according to claim 6,
wherein the protein comprises at least one selected from the group consisting of growth factors, hormones, interferons, vaccines, recombinant proteins, recombinant peptides, and enzymes.

8. The composition according to claim 7,
wherein the stabilization of the biological material prevents denaturation or aggregation of the biological material.

9. The composition according to claim 8,
wherein the denaturation or aggregation of the biological material occurs during a dehydration-rehydration process or a freeze-thaw process.

10. A method for stabilizing a biological material, comprising adding a composition for stabilizing a biological material comprising a CAHS (Cytosolic Abundant Heat-Soluble protein) protein which is represented by SEQ ID NO: 9 to the biological material.

11. The method according to claim 10,
wherein the CAHS protein is represented by SEQ ID NO: 2.

12. (canceled)

13. The method according to claim 1, wherein the mass ratio of the biological material to the CAHS protein is 1:2 or greater.

14. The method according to claim 13,
wherein the mass ratio of the biological material to the CAHS protein is 1:2 to 1:100.

15. The method according to claim 14,
wherein the biological material is any one selected from the group consisting of DNA, RNA, peptides, proteins, polysaccharides, and polypeptides.

16. The method according to claim 15,
wherein the protein comprises at least one selected from the group consisting of growth factors, hormones, interferons, vaccines, recombinant proteins, recombinant peptides, and enzymes.

17. The method according to claim 16,
wherein the stabilization of the biological material prevents denaturation or aggregation of the biological material.

18. The method according to claim 17,
wherein the denaturation or aggregation of the biological material occurs during a dehydration-rehydration process or a freeze-thaw process.

19. A composition for stabilizing a biological substance according to claim 1; a pharmaceutical formulation comprising a target biological substance.

20. The pharmaceutical formulation according to claim 19,
wherein the mass ratio of the target biological substance to the CAHS protein is 1:2 to 1:100.

21. (canceled)

22. (canceled)

23. (canceled)

24. (canceled)
